(19) United States
(12) Patent Application Publication
(10) Pub. No.: US 2012/0277700 A1
(43) Pub. Date: Nov. 1, 2012

(54) OSTOMY DEVICE

(75) Inventors: Alphonsus Joseph Amer, JR., Akron, OH (US); Himanshu S. Amin, Solon, OH (US); Michael E. Chesler, Chagrin Falls, OH (US)

(73) Assignee: FLOWCHECK MEDICAL DEVICES, LLC, Cleveland, OH (US)

(21) Appl. No.: 13/096,109

(22) Filed: Apr. 28, 2011

(51) Int. Cl. A61F 5/445

(52) U.S. Cl. 604/332

(57) ABSTRACT

The claimed subject matter relates to an ostomy seal applicator guide that facilitates application of the ostomy seal. The applicator can receive an absorbent material that can be employed during the seal application process to absorb moisture (e.g., waste matter seeping from the ostomy/stoma) from an ostomy, and facilitate maintaining clean, dry skin surrounding the ostomy so that the seal can be adhered to the patient's skin surrounding the ostomy area as well as properly align the seal to the ostomy area.

### BACKGROUND

A gastrointestinal (GI) tract begins with an esophagus (e.g., the tube connecting the mouth and stomach) and continues through a stomach, small intestine, large intestine (colon), rectum, and anus. Food is swallowed in the esophagus, mixed and churned in the stomach, and digested and absorbed in the small intestine. Waste leaves the small intestine as liquid. The colon absorbs water from the liquid material and stores the remainder as formed stool until it is passed voluntarily out of the body through the rectum.

A number of conditions associated with deficiencies of the GI tract for example may call for an ostomy as part of treatment and recovery. These conditions include cancer of the colon and rectum, trauma, malformations present from birth, obstruction of the bowel, complications of diverticulosis, and Crohn's disease. In each of these, an ostomy may a suitable form of treatment. In certain conditions the ostomy may be temporary and can be reversed at a later date.

An ostomy is a surgical procedure that creates an opening on an abdominal wall for waste products to move out of a body. The terms ostomy and stoma are general descriptive terms that are often used interchangeably though they have different meanings. An ostomy refers to the surgically created opening in the body for the discharge of body wastes. A stoma is the actual end of the ureter or small or large bowel that can be seen protruding through the abdominal wall.

An ostomy pouching system (also colloquially called a bag) is a medical device prosthetic that provides a means for collection of waste from a surgically diverted biological system (e.g., colon, ileum, urinary) and creation of a stoma. Pouching systems are most commonly associated with colostomies, ileostomies, and urostomies.

Pouching systems usually consist of a collection pouch bag, known as a one-piece system or, in some instances involves a mounting plate, commonly called a wafer or a baseplate, and a collection pouch that is attached mechanically or with an adhesive in an airtight seal, known as a two-piece system. The selection of systems varies greatly between individuals and is often based on personal preference and lifestyle. Ostomy pouching systems collect waste that is output from a stoma. The pouching system allows the stoma to drain into a sealed collection pouch, while protecting surrounding skin from contamination. Skin adhesion for modern wafers/baseplates typically consider five parameters required in an adhesive: 1) absorption, 2) tack and adhesion, 3) flexibility, 4) erosion resistance and 5) ease of removal. A wafer/baseplate may last between 4 to 10 days before it needs to be replaced; this is highly dependent on an individual's lifestyle, ostomy type, and anatomy. For maximum hygiene and to reduce risk of infection, it is recommended that a one-piece (open-end) bag should be changed every twenty-four hours. A two-piece bag's base plate should be changed weekly with the bag changed every twenty-four hours.

The method of attachment to the wafer varies between manufactures and includes permanent (one-piece), press-on/click (“Tupperware” type), turning locking rings and the recently introduced “sticky” adhesive mounts. The two-piece arrangement allows pouches to be exchanged without removing the wafer; for example, some people prefer to temporarily switch to a “mini-pouch” for swimming, intimate and other short-term activities. Mini-pouches are suitable for minimum usage only: one—two hour wear

Pouches can be broken down into two basic types: open-end (drainable) and closed-end (disposable). Open-end pouches have a re-sealable end that can be opened to drain contents of the pouch into a toilet. The end is a sealed velcro-type closure at the end that eliminates the need for a clip, the old-fashioned method of closure still employed by some manufacturers. Closed-end pouches must be removed and replaced with a new pouch once the bag is full. This system can cause extreme damage to the skin area under the adhesive due to the frequency of removal and re-application.

Application of pouches can be difficult especially for children and elderly people. The ostomy area can often be moist or wet as a result of seeping waste matter. Accordingly, it is desirable to maintain the ostomy area dry for proper application of adhesive associated with the pouch. Concurrently wicking the area, applying the adhesive, and fixing the pouch seal to the ostomy area can be a complicated and difficult task that often results in improper application of the seal which can lead to discarding the seal and reattempting to affix a new seal to the ostomy area. The seals are relatively expensive, and many insurance carriers will only compensate for two seals per day which is the amount of seals typically required for daily use. Discarding a seal can result in a patient incurring out of pocket expenses associated with utilizing extra seals.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The subject matter disclosed and claimed herein, in one or more aspects thereof, comprises an ostomy seal applicator guide that facilitates application of the ostomy seal. The applicator can receive an absorbent material that can be employed during the seal application process to absorb moisture (e.g., waste matter seeping from the ostomy/stoma). As noted supra, moisture around the ostomy/stoma is undesirable since can inhibit an adhesive from securing the seal to a patient's skin. The absorbent material draws and absorbs moisture from the ostomy/stoma area thus facilitating a clean dry skin surface for the seal to be secured to via the adhesive. Furthermore, the applicator facilitates guiding the seal for proper placement about the ostomy/stoma. Conventionally, a patient often attempts to apply the seal with adhesive applied thereon or on the skin periphery of the ostomy/stoma by using his/her hand in a manner similar to applying a bandage. Many times such attempts result in misalignment of the seal. Patients with an ostomy/stoma often are over the age of 60 and consequently may have diminished motor skills. The applicator allows for absorbing seeping waste matter as well as guiding the seal for proper alignment with the ostomy/stoma area. The applicator as described and claimed herein mitigates many of the foregoing issues (e.g., misalignment, diminished motor skills, seeping waste matter that can inhibit adhesion of the seal, and insurance only paying for a limited quantity of seals per day) associated with conventional ostomy seal application.

The following description and the annexed drawings set forth in detail certain illustrative aspects of the claimed subject matter. These aspects are indicative, however, of but a few of the various ways in which the principles of the claimed subject matter may be employed and the claimed subject matter is intended to include all such aspects and their equivalents. Other advantages and distinguishing features of the claimed subject matter will become apparent from the following detailed description of the claimed subject matter when considered in conjunction with the drawings.

### BRIEF DESCRIPTION OF THE DRAWINGS

FIG. 1 illustrates a diagram of an ostomy seal applicator, and absorbant material therein;

FIG. 2 depicts a diagram of the ostomy seal applicator with an ostomy seal thereon;

FIG. 3 depicts a diagram of the ostomy seal applicator with an ostomy seal affixed with an adhesive;

FIG. 4 illustrates the ostomy seal being guiding along the ostomy seal applicator;

FIG. 5 depicts a plunging apparatus for applying force on the absorbant material within the ostomy seal applicator;

FIG. 6 illustrates the plunging apparatus moving the absorbant material within and outside of the applicator;

FIG. 7 illustrates the seal being guided to an end of the applicator, and the absorbing material pushed to the end of the applicator;

FIG. 8 depicts an embodiment of the absorbant material with a draw string;

FIG. 9 illustrates the draw string employed to remove the absorbant material from the applicator;

FIG. 10 illustrates the plunging apparatus employed to remove the absorbant material from the applicator;

FIG. 11 depicts an embodiment of a flexible applicator;

FIG. 12 depicts an embodiment of a flexible applicator and flexible plunging apparatus.

FIG. 13 depicts in perspective view an embodiment of the seal being guided to an end of the applicator, and the absorbing material pushed to the end of the applicator so as to come into contact with an ostomy on the right side of an abdomen.

FIG. 14 depicts in perspective view an embodiment of the seal being guided to an end of the applicator, and the absorbing material pushed to the end of the applicator so as to come into contact with an ostomy on the left side of an abdomen.

FIG. 15 depicts the seal successfully applied around the ostomy.

FIG. 16 illustrates a pouch attached to the seal and configured to collect waste material that exits from the ostomy area.

### DETAILED DESCRIPTION

The claimed subject matter is now described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, well-known structures and devices are shown in block diagram form in order to facilitate describing the claimed subject matter.

The word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or.” Therefore, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

As used herein, the terms “infer” or “inference” generally refer to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

It is to be appreciated that the drawings depict various embodiments in schematic form, and thus are not necessarily to scale. Accordingly, the embodiments as described and claimed herein are intended to encompass any suitable size and configuration of components for effecting the various functionalities as described and claimed herein.

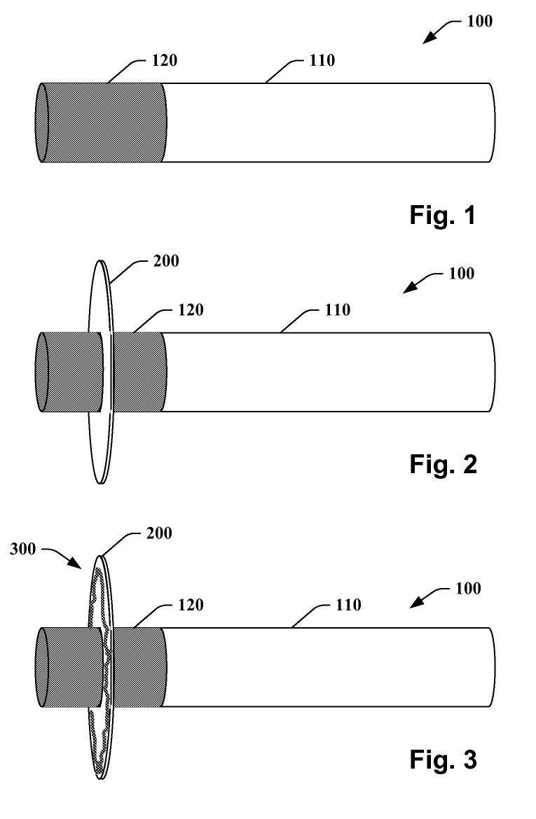

Referring now to the drawings, with reference initially to FIGS. 1-3, an ostomy seal applicator guide 100 is illustrated that facilitates application of an ostomy seal 200 (FIG. 2). The applicator 100 is configured to receive an absorbent material 120 that can be employed during a seal application process to absorb moisture (e.g., waste matter seeping from the ostomy/stoma) away from the ostomy and surrounding area. As noted supra, moisture around the ostomy/stoma is undesirable since it inhibits an adhesive 300 (FIG. 3) from securing the seal 200 to a patient's skin. The absorbent material 120 draws and absorbs moisture from the ostomy/stoma area thus facilitating a clean and dry skin surface for the seal 200 to be secured to via the adhesive. Furthermore, the applicator 100 facilitates guiding the seal 200 for proper placement about the ostomy/stoma. Conventionally, a patient often attempts to apply the seal 200 with adhesive applied thereon or on the skin periphery of the ostomy/stoma by using his/her hand in a manner similar to applying a bandage. Many times such attempts result in misalignment of the seal 200. Patients with an ostomy/stoma often are over the age of 60 and consequently may have diminished motor skills. The applicator 100 in combination with the absorbent material 120 allows for absorbing seeping waste matter as well as guiding the seal 200 for proper alignment with the ostomy/stoma area. The applicator 100 as described and claimed herein mitigates much of the foregoing issues (e.g., misalignment, diminished motor skills, seeping waste matter that can inhibit adhesion of the seal, and insurance only paying for a limited quantity of seals per day) associated with ostomy seal application.

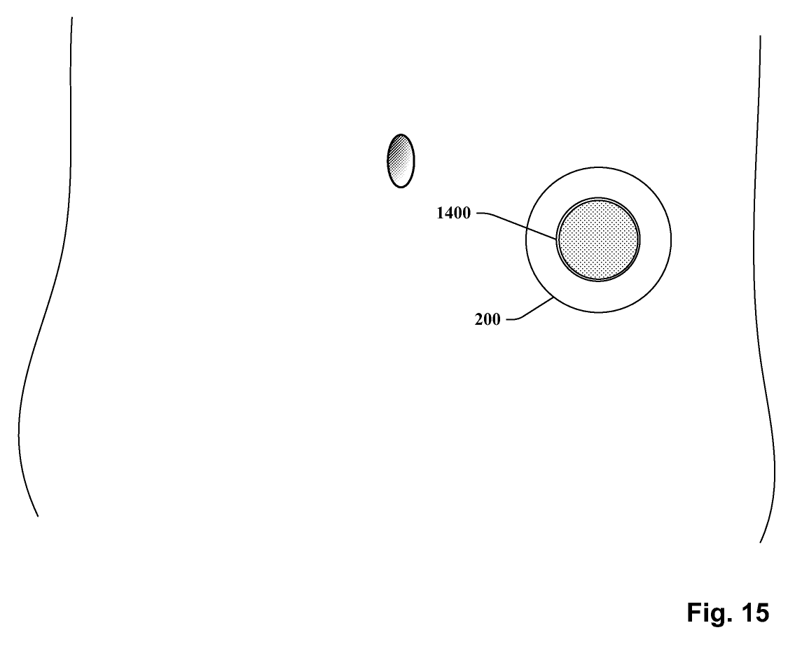

An embodiment of the applicator 100 has a tubular body 110. A front end portion of the applicator 100 is configured to receive the absorbent material 120. The circumference of the applicator 100 can be selected so as to match the inner circumferential opening of the seal 200 that is intended to expose the ostomy area so that waste matter can exit and be collected in a pouch 1500 (FIG. 15).

FIG. 3 illustrates the adhesive 300 applied to the seal 200. It is to be appreciated that the seal 200 can be positioned at any location on the applicator 100 suitable for a patient to apply the adhesive 300 to the seal. It is contemplated that in some cases a patient may opt to apply the adhesive 300 to the seal 200 prior to placing the seal 200 on the applicator 100.

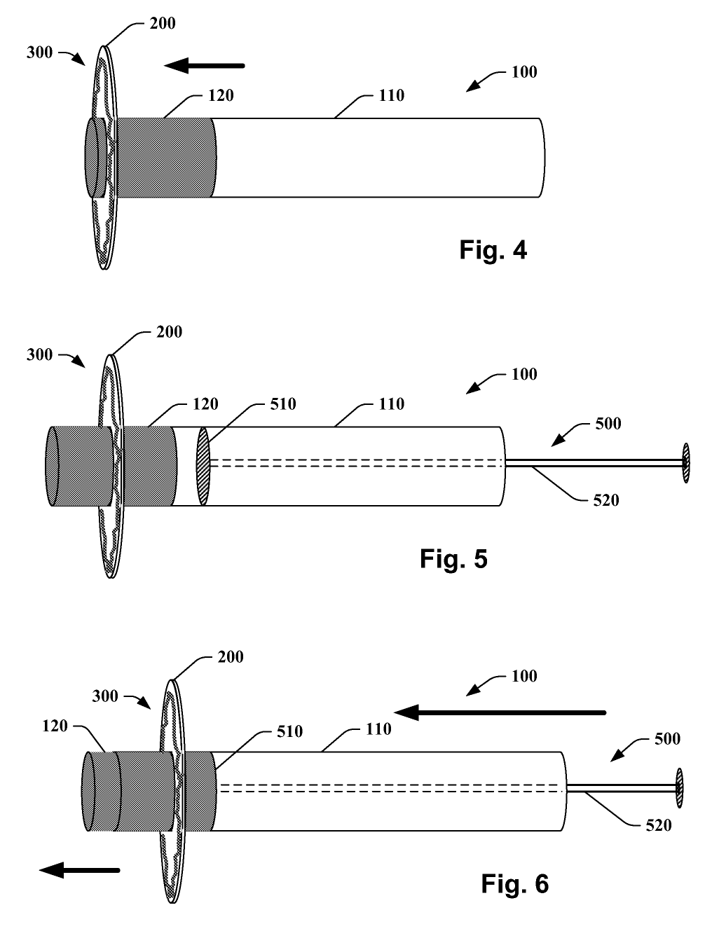

With reference to FIG. 4, matching the circumference of the applicator 100 to the opening of the seal facilitate sliding of the seal 200 along the length of the tubular body toward the ostomy area, and accurate alignment of the seal 200 around the ostomy. Furthermore, positioning the seal 200 on the tubular body 110 facilitates application of the adhesive 300 to a circumferential area 300 of an inner side of the seal 300 that is to come into contact with the patient's skin. Conventionally, a patient would often apply the adhesive to the seal and then attempt to attach the seal to the skin around the ostomy. However, misalignment can sometimes result in the adhesive coming into undesirable contact with the ostomy or stoma. Consequently, the seal might not be properly aligned with the ostomy, adhesion of the seal to the skin might be diminished as a result of the adhesive being degraded by coming into contact with waste matter. Moreover, such conventional technique for attaching the seal can be difficult for patients let alone those with diminished motor skills which can result in costly waste of seals since multiple attempts may be required in order to properly attach the seal. It is to be appreciated that sizing of the applicator 100 and opening of the seal 200 can vary as desired with a non-limiting goal of employing the applicator 100 to facilitate absorbing of moisture around the ostomy/stoma area in order to keep a patient's skin as dry as possible to facilitate adhesion of the seal to the skin, and so that the applicator 100 can be employed by the patient to guide the seal 200 for proper alignment and application. In an embodiment, the opening of the seal 200 is a sealing ring, and the tubular body 110 has a circumference that is within the range of 90 to 99.99 percent the circumference of the sealing ring of the ostomy seal. In another embodiment, the tubular body 110 has a circumference that is within the range of 80 to 99.99 percent the circumference of the sealing ring of the ostomy seal.

A variety of seals exist in the marketplace, and it is contemplated that the applicator can be configured and sized to accommodate many if not all available as well as foreseeable seal types and sizes. The tubular body 110 can comprise any material suitable for carrying out the functionalities described herein. For example, it is contemplated that plastic(s), composite(s), porcelain(s), metal(s), wood(s), rubber(s), polymers, laminate(s), as well as any other suitable material or respective combinations thereof can be employed in connection with the tubular body 110.

In yet another embodiment, the surface (inner or outer) can be coated to facilitate gliding the seal along the length of the applicator 100. In another embodiment, a coating (e.g., an anti-bacterial compound) can be employed that mitigates infection.

In an embodiment the absorbent material 120 is made of a highly liquid absorbable and expansible material such as for example cotton. In another embodiment, the absorbent material 120 can comprise absorbent compressed fibrous material. Accordingly, the absorbent material 120 can comprise any suitable absorbent material having acceptable absorbency or modulus of elasticity properties that is capable of absorbing or retaining liquid. The absorbent structure can be manufactured in a wide variety of sizes and shapes, and from a wide variety of liquid absorbing materials. For example, a representative, but non-limiting list of suitable materials includes cellulosic materials such as rayon, cotton, wood pulp, creped cellulose wadding, tissue wraps and laminates, peat moss, and chemically stiffened, modified, or cross-linked cellulosic fibers; synthetic materials, such as polyester fibers, polyolefin fibers, absorbant foams, e.g., a flexible resilient polyurethane foam, absorbent sponges, super-absorbent polymers, absorbent gelling materials; formed fibers, synthetic fibers, or any equivalent material, or combinations of materials, or mixtures of these.

In an embodiment, the absorbent material 120 can include a liquid absorbable envelope made of a web of material such as for example cotton gauze. Mesh material of silk, nylon, or the like can be employed. In yet another embodiment, the absorbant material 120 can include a medicant to mitigate infection. The absorbant material 120 can also comprise longitudinal grooves or spiral grooves to increase absorption capacity of the absorbant material 120.

FIG. 5 illustrates an embodiment of the applicator 100 that includes a plunger 500 that can be employed to apply pressure to the absorbant material 120 so as to cause it to move along the inner length of the tubular body 110 toward the ostomy as well as to continue applying pressure to the absorbent material 120 to maintain contact between the absorbent material 120 and the ostomy in order to facilitate absorbing of moisture from the ostomy area by the absorbent material 120. The plunger 500 comprises a gasket 510 that can be inserted into the tubular body 110 from the back end, and a plunger rod 520. By pushing the plunger rod 520 into the tubular body 110, the absorbent material 120 is advanced so as to come into contact with a patient's ostomy area. The gasket 510 can be made of elastomer, rubber, or any suitable material that facilitates a hermetic seal within the tubular body 110 so as to mitigate liquid from seeping through the back end of the tubular body 110. The plunger rod 520 can comprise any suitable rigid, or semi-rigid material such as for example: plastic, metal, wood, composite material, or the like. Thus, the plunger 500 functions in a manner similar to that of a plunger as employed in connection with a syringe to advance material within a tubular body.

FIG. 6 illustrates the plunger 500 being employed to advance the absorbent material 120 through the tubular body 110 toward the front end of the applicator 100.

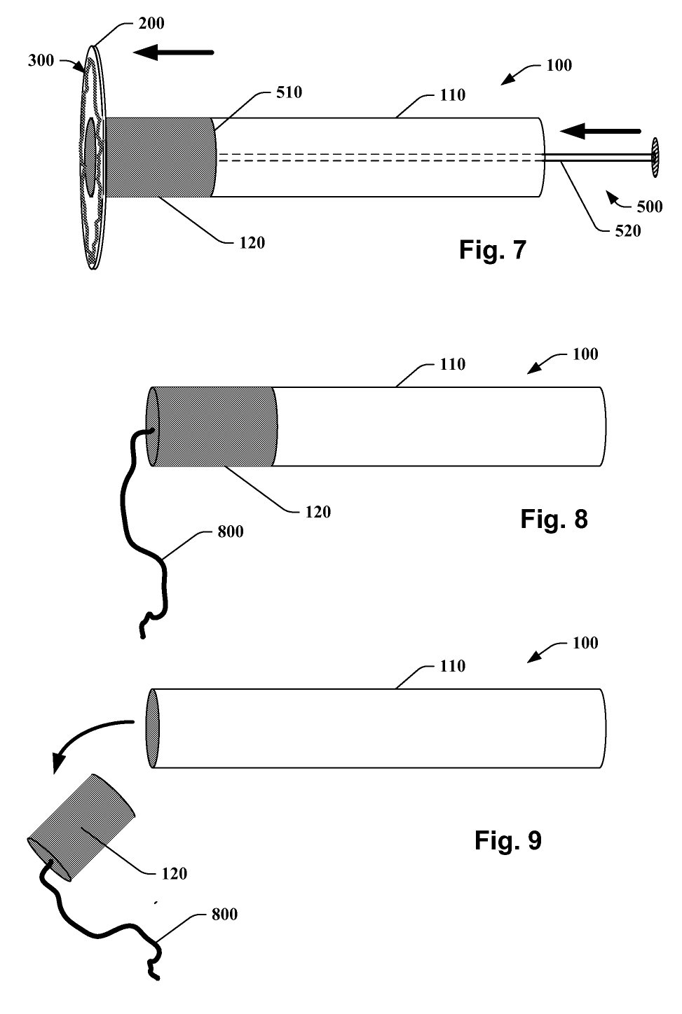

FIG. 7 illustrates the seal 200 with adhesive 300 thereon being guided along the outside of the tubular body 110 toward the front end of the applicator 100 which will be in contact with the patient's skin.

FIGS. 8 and 9 illustrate an embodiment where the absorbent material 120 includes a draw string 800 that facilitates removal of the absorbent material 120 from the applicator 100.

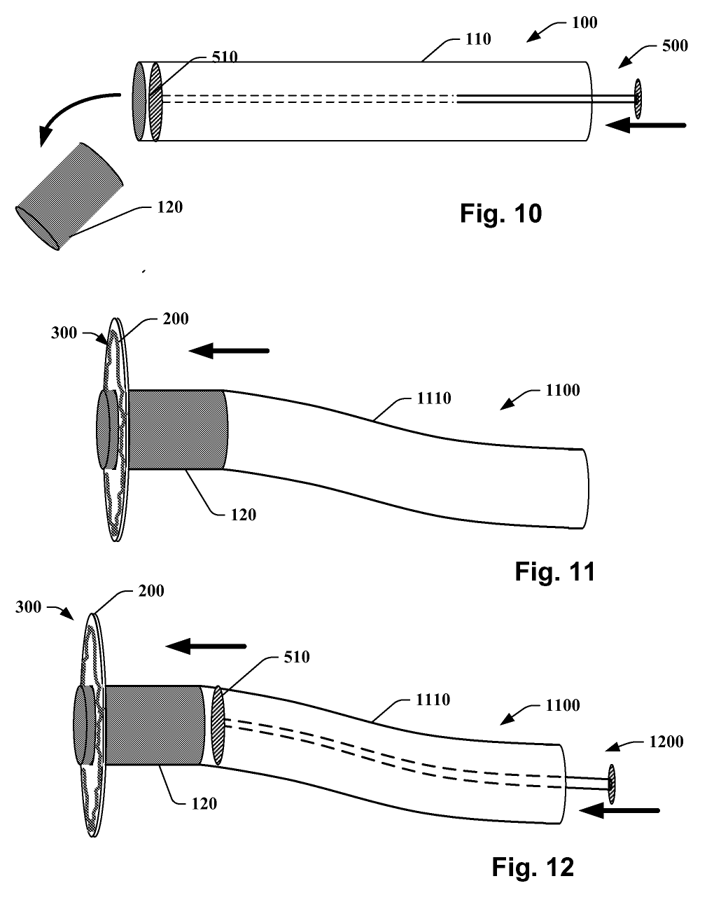

FIG. 10 illustrates en embodiment where the plunger 500 is employed to remove the absorbent material 120 from the front end of the applicator 100.

FIGS. 11 and 12 illustrate embodiments where the applicator 100 includes a flexible tubular body 1110 and a flexible plunger 1200.

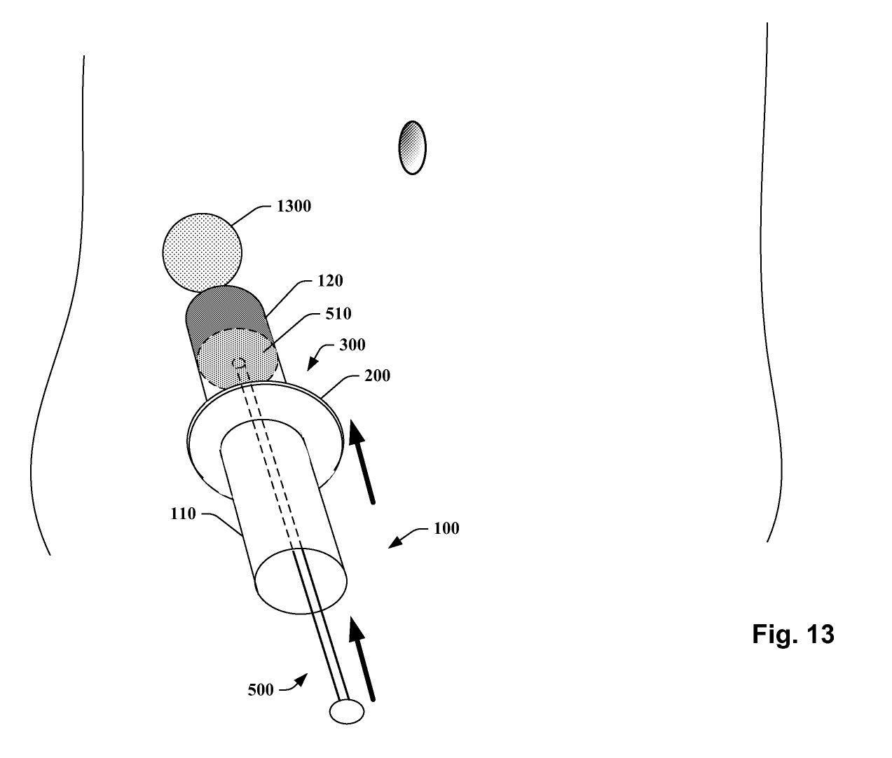

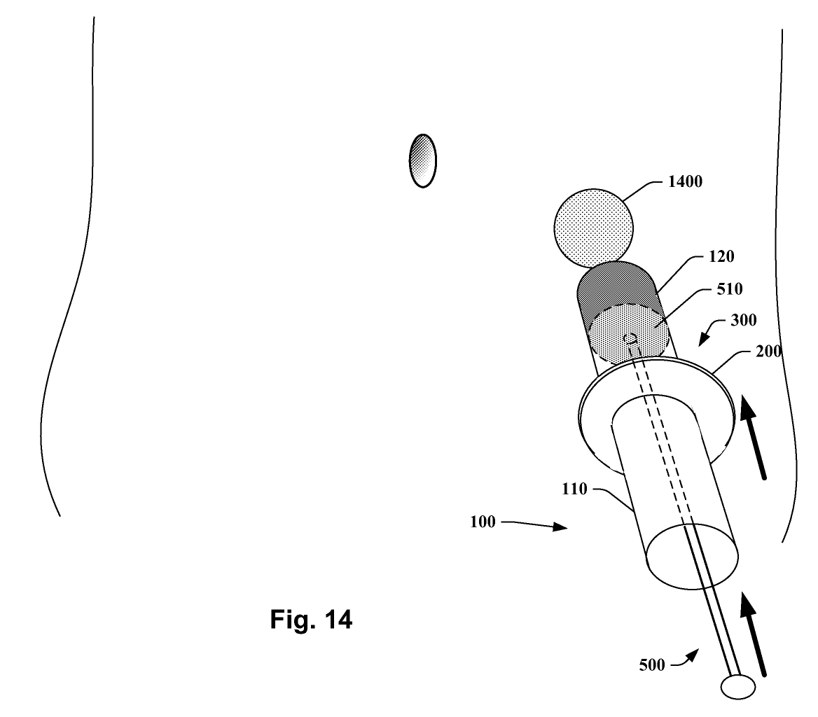

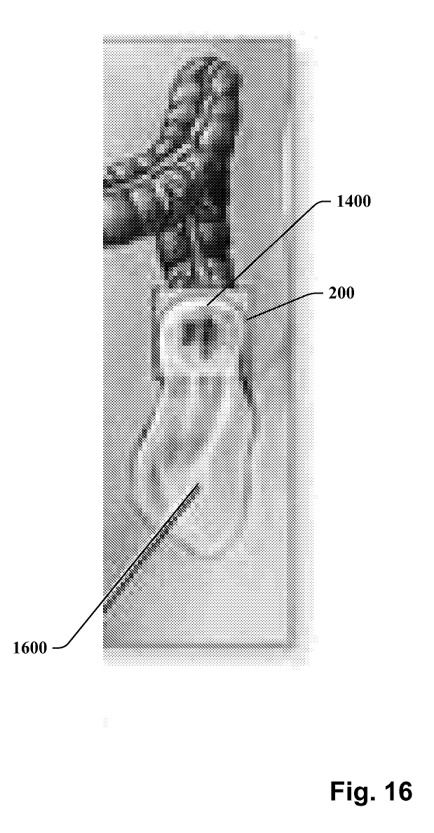

FIGS. 13 and 14 respectively depict in perspective view an embodiment of the seal being guided to an end of the applicator, and the absorbing material pushed to the end of the applicator so as to come into contact with an ostomy on the right, and left side of an abdomen. More particularly, one non-limiting methodology for employing the applicator 100 includes: inserting the absorbent material 120 into the applicator 100; inserting the tubular body 110 through an opening of the seal 200; applying an adhesive to a front surface of the seal 200 which will come into contact with a patient's skin; aligning the tubular body 110 with the patient's ostomy so that the front end of the tubular body is in circumferential contact with the ostomy, and the absorbent material is positioned to come into contact with the ostomy area; apply pressure to the plunger 500 so that it advances the absorbent material 120 so as to come into contact with the ostomy area, and absorb moisture from the ostomy area; if moisture exists on the skin area surrounding the ostomy wipe the moisture away while maintaining pressure of the absorbent material 120 against the ostomy to mitigate additional moisture seeping onto the patient's skin surrounding the ostomy; optionally clean the skin surrounding the ostomy with an alcohol wipe of the like to facilitate drying of the skin; once the skin area that is to come into contact with the seal is sufficiently clean and dry, manually guide the seal along the applicator toward the patient while maintaining pressured contact of the absorbent material to the ostomy area; position the seal 200 to the front end of the applicator 110 and manually apply pressure to the seal 200 so that it comes into contact with the patient's skin, and continue applying pressure to the seal 200 so that it properly attaches to the patient's skin surrounding the ostomy area; once the seal has been attached to the patient's skin in a suitable manner while still maintaining pressure on the seal 200 toward the patient's skin, pull the applicator 100 away from the patient's body; remove the absorbent material 120 from the applicator 100 (e.g., via the draw string or plunger 500); and apply an ostomy pouch 1600 (FIG. 16) to the seal 200.

FIG. 15 depicts the seal 200 successfully applied around the ostomy.

FIG. 16 illustrates a pouch 1600 attached to the seal 200 and configured to collect waste material that exits from the ostomy area.

What has been described above includes examples of the various embodiments. It is, of course, not possible to describe every conceivable combination of components or methodologies for purposes of describing the embodiments, but one of ordinary skill in the art may recognize that many further combinations and permutations are possible. Accordingly, the detailed description is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the embodiments. In this regard, it will also be recognized that the embodiments includes a system as well as a computer-readable medium having computer-executable instructions for performing the acts and/or events of the various methods.

In addition, while a particular feature may have been disclosed with respect to only one of several implementations, such feature may be combined with one or more other features of the other implementations as may be desired and advantageous for any given or particular application. Furthermore, to the extent that the terms “includes,” and “including” and variants thereof are used in either the detailed description or the claims, these terms are intended to be inclusive in a manner similar to the term “comprising.”

What is claimed is:

1. An ostomy seal applicator, comprising:
a tubular body configured to receive an absorbent material through a first or second end of the tubular body, the tubular body configured to receive along its outer periphery an ostomy seal through the first or second end of the tubular body so that the ostomy seal can be advanced along the outer periphery of the tubular body toward the first end.

2. The ostomy seal applicator of claim 1 wherein the first end of the tubular body is configured to be applied against an ostomy area, and the absorbent material configured to be placed in contact with the ostomy area absorb moisture from the ostomy area, and the ostomy seal advanced along the tubular body to be adhered to skin of the patient surrounding the ostomy area.

3. The ostomy seal applicator of claim 2, comprising a plunger that is configured to apply pressure to the absorbent material so that it advances within the tubular body toward the first end.

4. The ostomy seal applicator of claim 3, wherein the plunger is configured to apply pressure to the absorbent material so that the absorbent material maintains pressured contact with the ostomy area to facilitate the absorbent material absorbing moisture from the ostomy area.

5. The ostomy seal applicator of claim 3, the plunger comprising a gasket.

6. The ostomy seal applicator of claim 5, the gasket comprising rubber or an elastomer material so as to mitigate moisture exiting the second end of the applicator.

7. The ostomy seal applicator of claim 1, the tubular body comprising at least one of wood, metal, plastic, polymer, or composite material.

8. The ostomy seal applicator of claim 1, wherein the tubular body is rigid.

9. The ostomy seal applicator of claim 1, wherein the tubular body is flexible.

10. The ostomy seal applicator of claim 1, wherein the tubular body is semi-rigid.

11. The ostomy seal applicator of claim 1, wherein the tubular body has a circumference that mates with sealing ring of the ostomy seal.

12. The ostomy seal applicator of claim 1, wherein the tubular body has a circumference that is within the range of 90 to 99.99 percent the circumference of a sealing ring of the ostomy seal.

13. The ostomy seal applicator of claim 1, wherein the tubular body has a circumference that is within the range of 80 to 99.99 percent the circumference of a sealing ring of the ostomy seal.

14. An ostomy seal application system, comprising:
an absorbent material;
a tubular body configured to receive the absorbent material through a first end of the tubular body, the tubular body configured to guide along its outer peripheral area an ostomy seal; and
a plunger that is employed to advance the absorbent material toward the first end of the tubular body, and maintain pressured contact between the absorbent material and an ostomy.

15. The system of claim 14, the tubular body having a circular cross-sectional area.

16. The system of claim 14, the tubular body having an elliptical circular cross-sectional area.

17. The system of claim 14, the tubular body having a rectangular or square circular cross-sectional area.

18. The system of claim 14, the tubular body having a polygonal cross-sectional area.

19. The system of claim 14, the tubular body comprising at least one of: plastic(s), composite(s), porcelain(s), metal(s), wood(s), rubber(s), polymers, or laminate(s).

20. The system of claim 14, the absorbent material comprising at least one of rayon, cotton, wood pulp, creped cellulose wadding, tissue wraps, laminates, peat moss, chemically stiffened, modified, or cross-linked cellulosic fibers; polyester fibers, polyolefin fibers, absorbent foams, flexible resilient polyurethane foam, absorbent sponges, super-absorbent polymers, absorbent gelling materials; formed fibers, synthetic fibers.

21. The system of claim 14, the absorbent material comprising a medicant.

22. The system of claim 14, the absorbent material is shaped to slide along an inter periphery of the tubular body.

23. The system of claim 14, the ostomy seal comprises an ostomy seal ring that is configured to slide along the outer periphery of the tubular body in an axial direction such that the ostomy seal can be manually advanced toward and aligned with the ostomy.

24. A method for applying an ostomy seal comprising:
inserting an absorbent material into a tubular body;
slidably attaching an ostomy seal to an outer periphery of the tubular body;
aligning the tubular body with an ostomy;
maintaining pressured contact between the absorbent material and the ostomy; and
sliding the ostomy seal along an axial direction of the tubular body towards the ostomy; and
adhering the ostomy seal to skin surrounding the ostomy.
